(19) United States
(12) Patent Application Publication
(10) Pub. No.: US 2017/0098053 A1
(43) Pub. Date: Apr. 6, 2017

(54) TELEGENETICS

(71) Applicant: Georgetown University, Washington, DC (US)

(72) Inventors: Abhishek Pandey, Rockville, MD (US); Howard Federoff, Bethesda, MD (US); Ophir Frieder, Chevy Chase (MD)

(73) Assignee: Georgetown University, Washington, DC (US)

(21) Appl. No.: 15/315,098

(22) PCT Filed: Jun. 8, 2015
(86) PCT No.: PCT/US15/34735

§ 371 Date: Nov. 30, 2016

(51) Int. Cl. G06F 19/00; G06F 19/28; G06F 19/18; A61B 5/00

(52) U.S. Cl. CPC G06F 19/3418; A61B 5/0022; G06F 19/322; G06F 19/28; G06F 19/18

(57) ABSTRACT

Disclosed herein are systems and methods that utilized a network of dispersed computing systems, specialized software, and other networking technology for providing remote communications for facilitating genetics counseling services between people or devices located at remote locations. The disclosed technology can utilize a centralized computing system including a central server system and one or more data warehouses. The centralized computing system can interact with various remote client computing devices to enable remote communications between patients and healthcare professionals. The communications can be secured, encrypted, recorded, transcribed, and/or stored in an editable, parsable, searchable, and shareable format. The systems and methods can also facilitate automated scheduling, automated billing, requesting of genetic testing, storing of genetic testing data, analysis of genetic testing data, suggested treatments, provision of educational literature, actual treatment, and/or other features.

### CROSS-REFERENCE TO RELATED APPLICATIONS

This application claims the benefit of U.S. Provisional Application No. 62/009,819, filed on Jun. 9, 2014, and also claims the benefit of U.S. Provisional Application No. 62/015,896, filed on Jun. 23, 2014, both of which are incorporated by reference herein in their entirety.

### FIELD

This disclosure relates to the provision of genetic testing and/or genetic counseling services between remote locations.

### BACKGROUND

Telemedicine is generally considered to be the use of telecommunication technologies to provide health care services at a distance. For example, a patient may consult with a doctor or other clinician over the telephone or online instead of meeting in person. However, scheduling the telecommunications, taking notes of the communication, facilitating laboratory testing, providing diagnoses/treatments/referrals based on the communications, enabling billing, and other related procedures are typically performed in a more conventional manner that is not integrated with a telemedicine system.

Genetic testing and genetic counseling allows for the determination of a patient's genetic characteristics, diagnosis of genetic polymorphisms, mutations, epigenetic changes or other irregularities and consequential health vulnerabilities, identification of family relationships, estimation of likelihood of future diseases or conditions, prenatal testing, provision or risk reduction or other treatment strategies, and other useful applications. Typically, a patient submits a DNA sample for genetic testing and then consults with a genetic counselor regarding the testing results and its ramifications.

### SUMMARY

Disclosed herein are systems and methods for providing improved genetic testing and genetic counseling services between or among remote locations. The disclosed technology can utilize a centralized computing system including a central server system and one or more data warehouses. The centralized computing system can interact with various remote client computing devices to enable remote communications between patients and healthcare professionals. The communications can be secured, encrypted, recorded, transcribed, translated, and/or stored in an editable, parsable, searchable, minable, and shareable format. The systems and methods can also facilitate automated scheduling, automated billing, automated suggesting or human requesting of genetic testing, storing of genetic testing data, analysis of genetic testing data, suggested treatments, provision of educational literature, automated notification of potentially relevant new developments, and/or other features.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 illustrates an exemplary system for implementing the disclosed telegenetics technology.

FIG. 2 is a flow chart illustrating an exemplary telegenetics method.

FIG. 3 illustrates an exemplary computing environment for implementing the disclosed telegenetics technology.

### DETAILED DESCRIPTION

Telegenetics can be facilitated by a system that includes a centralized computing system that interacts with remotely located client computing devices, which are accessed by patients, referring medical practitioners, and genetics healthcare providers located in different places. The term “telegenetics,” as used herein, means the provision of genetic testing and/or genetic counseling services between two or more remote locations, such as using a centralized computing system that communicates with one or more client computing devices over a remote communications network.

The centralized computing system can include any number of discrete computing devices, including one or more computing devices or networks that function as a server (referred to herein collectively as “the central server”) and one or more computing devices that function as databases or data warehouses (referred to herein collectively as “the data warehouse”). The central server and the database include communication mechanisms to communicate with each other and/or remote computing devices and memory to store data thereon. The centralized computing system can receive, store, parse, analyze, and provide various forms of data related to genetic testing and genetic counseling for any number of patients.

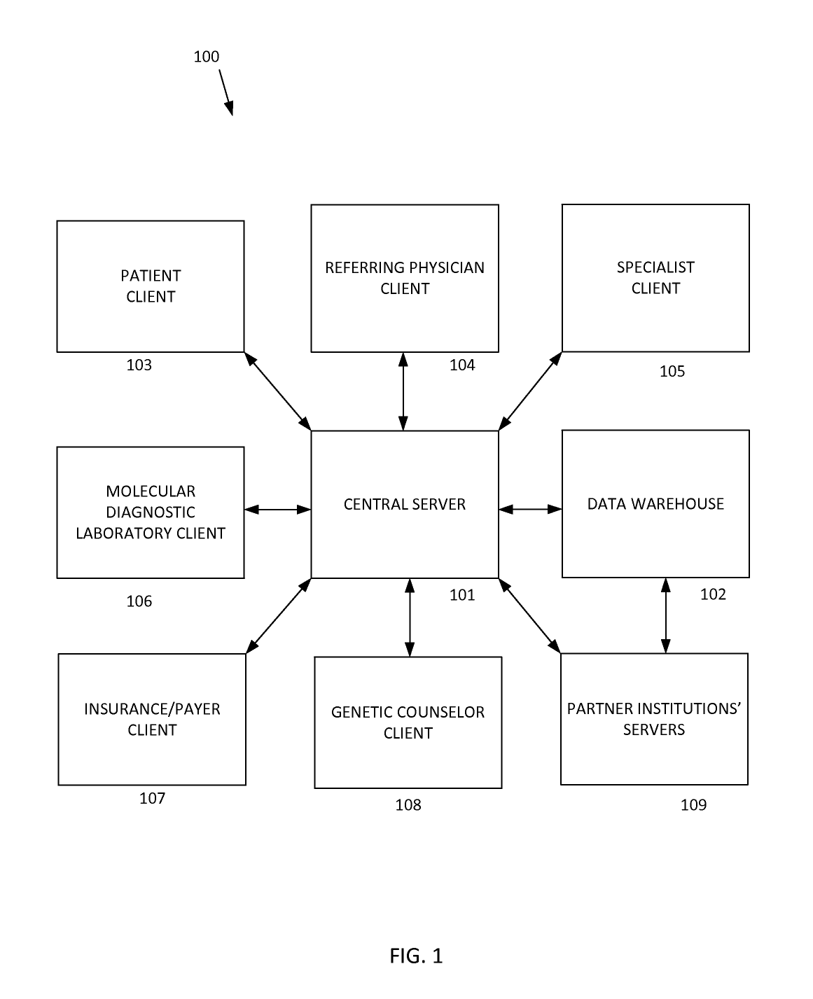

FIG. 1 illustrates an exemplary system architecture 100 for implementing the disclosed telegenetics technology. The arrows between the various blocks indicate electronic communications pathways, such as via Internet connections, wireless networks, cellular networks, local area networks, direct wired connections, or otherwise. The system includes a central server 101 and a data warehouse 102 that form a logically centralized computing system. It is within the scope of the disclosed technology that the centralized computing system can be implemented as a distributed platform, such as described herein. The central server 101 and data warehouse 102 can be located together (e.g., part of the same machine) or remote from each other. Further, the central server 101 and the data warehouse 102 can be owned by different entities. For example, the central server 101 may be owned by a public web services provider, such as Amazon Web Service or the like, while the data warehouse 102 may be owned by another entity that provides data storage, such as a public web storage provider.

The data warehouse 102 can comprise a central repository that can store and integrate various forms and classes of data from different sources. The data warehouse 102 can be configured to receive and store patient electronic medical records, which can include any information related to a patient, including general medical information such as a patient's location, age, gender, family relationships, previous medical history and current medical conditions, as well as genetic-specific information such as raw genetic data, sequencing data, epigenetics, single nucleotide polymorphism (SNP) data, other genetic testing results, reports based on genetic testing results, communications between the patient and the referring medical practitioners and/or the genetics healthcare providers, diagnoses, and associated treatment plans. The data warehouse 102 can also store data relating to medical literature, educational content, locations of facilities, scheduling information related to facilities and people, and various other information useful in facilitating telegenetics. Examples of medical literature that can be accessed include Pub Med from the National Library for Biotechnology Information, dbSNP from the National Center for Biotechnology Information, the Genetic Association Database, OMIM (Online Mendelian Inheritance in Man) database of human genes and genetic phenotypes, SNPedia, ClinVar, and the Free the Data initiative of California HealthCare Foundation. Medical literature can also be obtained from proprietary databases and knowledgebases. Information obtained from such sources can be condensed and/or reduced to make it more efficiently usable for clinical decision making.

Referring again to FIG. 1, the system 100 can also include various client computing devices that communicate with the central server 101 and/or with other of the client computing devices. These can include any number of patient clients 103, referring physician or primary care physician clients 104, specialist clients 105, molecular diagnostic laboratory (MDL) clients 106, insurance/payer clients 107, genetic counselor clients 108, and/or other clients. Any of the client devices can communicate directly with the central server, and/or may communicate with other client devices directly (i.e., bypassing the central server) or via the central server.

The patient client 103 can comprise any computing device used by a patient to access or communicate with the central server 101. The patient server 103 can comprise a personal computer, a mobile computing device, a public computing device, or any other device having access to a communications pathway for transmitting information to and from the central server 101. In one example, the patient client 103 can be a patient's cellular phone that includes a software application for accessing the central server 101 via the Internet. In another example, the patient client 103 can be a desktop computer that accesses a website portal for accessing the central server via a web browser and the Internet.

The referring physician client 104 can comprise any computing device having access to the central server 101 and that is used by any clinician or primary care provider who assumes medical care for the patient, such as a licensed and/or board certified physician, nurse practitioner, or physician's assistant.

The specialist client 105 can comprise any computing device having access to the central server 101 and that is used by a specialist who provides input to or receives output from the patient, the referring physician, genetic counselor, or other medical provider related to the patient. Specialists can include, but are not limited to, those who practice surgical oncology, medical oncology, obstetrics/gynecology, neonatology, neurology, pediatrics and its subspecialities (e.g. cardiology), medical genetics, or nursing staff associated with the relevant specialist. In some examples, the specialists are board-certified physicians other than internal medicine, neurology, pediatrics, pyschiatry or family medicine, or practice in a specialty recognized as being other than primary care.

The genetic counselor client 108 can comprise any computing device having access to the central server 101 and that is used by a genetic counselor for the patient. The term “genetic counselor” refers to a person who counsels the patient regarding genetics and genetic testing data, and includes, but is not limited to, a certified genetic specialist, a medical geneticist, or other genetic counselor serving the patient.

The molecular diagnostic laboratory (MDL) client 106 can comprise any computing device having access to the central server 101 and that is used by an MDL. An MDL includes, but is not limited to, any entity that receives a patient's biomedical specimen (e.g., saliva, blood, amniotic fluid, or tissue of a patient), performs genetic testing on the specimen, and produces patient genetic testing results for the patient based on the testing. For example, the genetic testing results could include a full genomic sequence, a sequence of a particular gene, a portion of a gene sequence, and/or identification of a single nucleotide polymorphism across the genome or at a particular genetic locus or genome-wide or gene/locus specific epigenetic analysis. Results could be obtained from a DNA sequence device, protein expression analysis, DNA methylation profile, and/or DNA array such as those available from Affymetric, Inc., Illumina, GE Healthcare, Applied Biosystems, Beckman Coulter, Eppendorf Biochip Systems, and Agilent. In some examples, genetic results include identification of epigenetic features of the genome such as DNA methylation or chromatin remodeling, for example as determined by bisulfite conversion or DNA methylation enrichment.

The insurance/payer client 107 can comprise any computing device having access to the central server 101 and that is used by an insurance company, an agent of an insurance company, or other payment or billing related entity. The insurance/payer client 107 can send and receive any data related to a patient's medical activities, such as patient data, ICD codes, diagnosis codes, procedure codes, and/or other medical billing information related to treatment of the patient.

The data stored in the centralized computing system may also be accessed and/or shared with various partner institutions' servers 109 and/or other external computing systems that have the appropriate permissions. Similarly, the centralized computing system may access information stored by the partner institutions' systems. Such collaborative sharing of medical information can improve analytics and empirical determinations by increasing the total data available for analysis, which can lead to more accurate diagnosis and more effective treatment plans and medical advice.

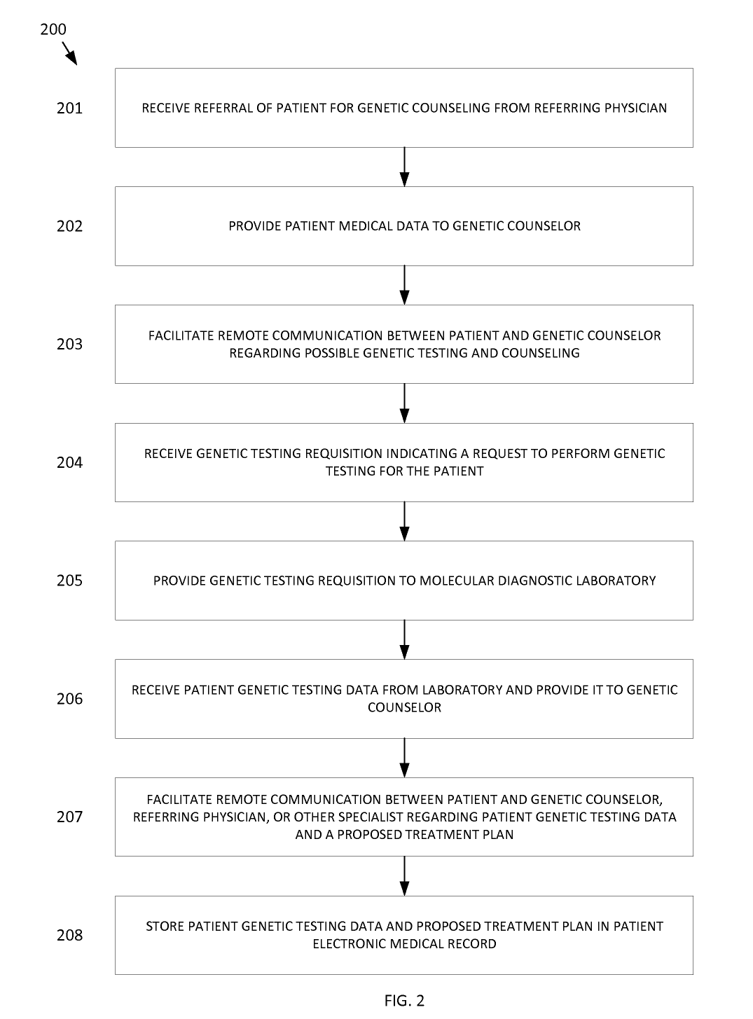

Various telegenetics methods and process are described below with reference to the exemplary method 200 of FIG. 2 and the system 100 and its various components of FIG. 1.

Generally, a patient is initially evaluated by a referring clinician such as a physician who determines that the patient's circumstances may warrant genetic counseling. In such a case, the referring physician may refer the patient to a genetic counselor for genetic counseling. At block 201 of FIG. 2, the centralized computing system receives a referral of the patient for genetic counseling from the referring physician. This can be accomplished, for example, by the referring physician or his agent interfacing with a referring physician client 104 and sending the referral electronically to the central server 101. The central server 101 may then provide the referral to the genetic counselor, such as via the genetic counselor client 108. In other embodiments, the referring physician may provide the referral directly to the genetic counselor without using the system 100, or can provide a referral note to the patient, who can then provide it to the genetic counselor.

In some embodiments, the patient must have a referral from a licensed medical practitioner for genetic counseling before seeing the genetic counselor, such as to avoid injudicious use of medical resources, particularly when counseling is likely not necessary or useful. The medical practitioner providing the referral will consider such factors as the likelihood of a genetic explanation for a medical condition, the likelihood that a genetic evaluation would be therapeutically beneficial or cost-effective, the reliability of genetic information that a patient has obtained from other sources, and the clinical severity of the patient's condition.

At block 202, the central server 101 can then provide relevant patient medical data electronically to the genetic counselor, such as via the genetic counselor client 108. Such patient medical data may be stored in the data warehouse 102. The genetic counselor may then review the patient medical data prior to consulting with the patient.

The patient and the genetic counselor can then arrange an appointment to communicate regarding genetic counseling. Information regarding locations and availability of the patient and/or the genetic counselor may be provided to and stored in the centralized computing system, and used to automatically suggest mutually appropriate appointment times when the patient and the genetic counselor are both available to communicate with each other. In some embodiments, the system and/or the physicians can consider the urgency of counseling for patients when determining scheduling, with more urgent cases being scheduled with higher priority. For example, patients with higher immediate medical risks and/or stress-inducing conditions can be scheduled and treated sooner or with higher priority. For example, a pregnant woman with a history of past spontaneous abortions or fetal abnormalities could be urgently referred for genetic evaluation as early as possible in the pregnancy to identify genetic conditions (such as methylenetetrahydrofolate reductase, MTHFR, mutations in the mother) that could be identified and readily addressed by nutritional therapy.

Such automated scheduling can include selecting locations where each participant can be located during the communication and/or clients that each participant can use for the communication. The patient and the genetic counselor may be located at any two locations that are connected with some kind of communications link. The system can select an appropriate office or facility having a genetic counselor client 108 that the genetic counselor can use and that is available during the selected time. The patient may similarly specify location and the type of patient client 103 to be used, including the computing and communications specifications to be used.

The selection of appointment times and locations can also be determined based on electronic calendar programs for each party, such as calendar programs that run on the client devices or the individual's personal phones or computers that are linked to the system 100.

In some embodiments, scheduling of facilities, locations, and/or times can be done based at least in part on patient insurance coverage criteria. For example, a facility can be selected in part because the costs associated with using that facility is included in the patient's insurance coverage.

At block 203, the central server 101 facilitates any number of remote communications between the patient and the genetic counselor regarding genetic counseling and possible genetic testing for the patient. The central server 101 can enable remote communications between the patient client 103 and the genetic counselor client 108. The remote communications can comprise various types of communication means, such as Voice-over-Internet-Protocol (VoIP), other live or recorded voice communications, text messaging such as via a chat window, recorded audio-video communications using video and audio recorders at one or both of the client devices, live two-way audio-video communications using video and audio recorders at both of the client devices (e.g. Skype®), or other remote communication means. Any such communications can be transmitted in a secure and/or encrypted manner, and the client devices can similarly be protected with passwords and other security to prevent unauthorized access. This can provide for compliance with privacy regulations, such as under HIPAA.

These remote communications can pass through the central server 101 and while being communicated between the client devices. The central server 101 can capture, copy, record, transcribe, translate, and/or otherwise obtain communication data from the communications, and can store the obtained data in the data warehouse 102. For example, text communications can be logged directly as written, can be translated into different languages, and/or can be converted to an audio/voice format. Support for the disabled (e.g., hard of hearing or suffering from vision loss) can also be provided. Video data may be recorded or captured and stored for later replay. Audio data may be recorded and stored as audio files and/or transcribed into text and stored as text. Any such data that are stored may be presentable in a visual format on a client device for later selection and review.

The data obtained from the remote communications can be stored in the data warehouse 102 in a secured and/or encrypted format to prevent unauthorized access. Role based access schemes can be used to further control access to sensitive data, such as is described in U.S. Pat. No. 8,271,527 entitled “Refined Permission Constraints Using Integral and External Data Extraction in a Role-Based Access Control System,” which is incorporated herein by reference. The data can be stored in one or more data formats. The data formats can be selected to be human readable, machine readable, digitally parsable, searchable, editable, sharable, scalable, and/or updatable. In some examples, the data formats can include data objects consisting of attribute-data pairs. In some examples, the data formats can be language-independent. Exemplary data formats can include JavaScript Object Notation (JSON) and Extensible Markup Language (XML).

In some embodiments, the data stored in the data warehouse 102 can be logged for safety, auditing, and/or billing purposes.

In some embodiments, the stored communications data can be accessed, reviewed, replayed, edited, annotated, and/or commented upon by any of the people involved in the communication and/or by other people associated with the system, such as system administrators. Permitted auditors can likewise access the data. For example, the patient may review a transcript of a VoIP call with the genetic counselor, and may edit typos, correct misstatements or other errors, add supplemental information or comments, or delete portions of the transcript. Such changes can be reviewable to ensure compliance with medical practice and regulations.

The remote communications can be live, or can be recorded for later review by the other party. In some embodiments, an application or website on the patient client can present questions or prompts to the patient and then record the patient's answers for later review by the genetic counselor. For example, the application or website can present an avatar representing the genetic counselor that asks the patient questions. The avatar may then “listen” to the patient's answers while they are recorded. The avatar may look like the genetic counselor, such as by presenting an image of the genetic counselor on the patient's screen. Alternatively, the avatar may be an identifier associated with the role of the genetic counselor but not the counselor's particular identity. For example, the avatar may be an image suggestive of a genetic counselor, such as a double helix.

In some embodiments, the patient client can capture images and/or video of the patient, such as the patient's face, and transmit them to the central server along with audio and/or text that the patient provides during the communications. The images/video of the patient can be viewed by a physician and/or counselor to help determine the patient's physical characteristics, health, state of mind, etc. For example, certain physical characteristics of the patient, such as dysmorphic features or unusual pigmentation, may indicate particular genetic characteristics or syndromes of the patient, and can help determine whether and what kind of genetic testing may be needed. Such images can be used to suggest additional diagnostic/genetic possibilities to supplement primary observations and findings made by a referring physician. Images may also be obtained by specialists and transmitted apart from an external view of the patient to provide diagnostic information about organs that cannot be readily ascertained from external inspection. For example, retinal photographs may be obtained by an ophthalmologist or ophthalmic technician, or video images from colonoscopy may be obtained from a gastroenterologist and transmitted to the counselor. Any data that are relevant may be used, including radiographic images, dynamic studies, and other information available from a picture archiving and communication system (PACS).

Based on the information provided by the patient during the remote communications, along with other patient medical information, the genetic counselor can determine whether or not to request genetic testing for the patient. The genetic counselor may also seek input from the referring physician and/or one or more specialists in evaluating the patient. Such communications can comprise remote communications using the system 100 wherein the communications are made using the client devices 103, 104, 108 via the central server 101. All of the description herein relating remote communications involving the patient can also apply to communications between the genetic counselor, the referring physician, specialists, or other medical personnel. For example, such communications can be encrypted, stored, edited, and later accessed.

The genetic counselor or referring physician may then request genetic testing for the patient. At block 204 of FIG. 2, the central server 101 receives a genetic testing requisition indicating a request to perform genetic testing for the patient. This testing request can be submitted by the genetic counselor or referring physician via the respective client device, such as by filling out an electronic form or clicking buttons on a user interface. The requisition can be stored in the data warehouse 102.

At block 205, the central server 101 provides the genetic testing requisition and any other relevant patient data to a MDL for performing the genetic testing. Such transmission can be secure and/or encrypted. The particular MDL can be selected by the system based on availability, proximity to the patient, cost, services offered, and/or other criteria. The MDL or an associated clinical laboratory may be located near the patient so that the patient can personally visit the MDL or clinical laboratory and provide a biological sample, or the patient may mail or otherwise send a biological sample to the MDL. The referring physician may also obtain the sample at the referring physician's office or clinic after remote consultation with a genetic counselor or specialist. The referring physician may then send the sample to the MDL on behalf of the patient. The MDL can then carry out the genetic testing using the biological sample and produce genetic testing data based on the patient's biological sample.

Based on the genetic testing data, the MDL can create a curated genetic testing report, which may include an analysis of the testing data. The MDL can then provide the curated genetic testing report along with files containing the raw genetic testing data, such as in variant call format (VCF) that includes meta-information lines, to the central server 101 to be stored in the data warehouse 102.

At block 206 of FIG. 2, the central server 101 receives the genetic testing data and/or reports for the patient (including the testing report and/or the raw data) from the MDL and provides it to the genetic counselor and/or to other medical personnel related to the patient's care. The genetic testing data may alternatively be obtained from any other source, and may already be prepared and stored, then obtained upon request and provided to the genetic counselor or other personnel. The genetic counselor can then review the genetic testing data and reports and can optionally consult with the referring physician or specialists regarding the patient's case to develop diagnoses and/or a proposed treatment plan for the patient.

In some embodiments, the system 100 can provide an electronic visual display or representation of the patient genetic testing data that indicates relevant mutations in interrogated genes of the patient. For example, the visual display can include a representation of each of the patient's 46 chromosomes with a marker on the interrogated genes. Alternatively, the display can be a browser into which a search term such as the name of a gene, medical condition, medication, gene product, physical finding, mutation, or polymorphism can be entered to determine if any of the genetic testing data are associated with the search terms.

In some embodiments, the system 100 can provide current medical literature to the genetic counselor or other medical professionals related to the patient's case. The medical literature can assist the medical professionals in more accurately diagnosing the patient's medical condition and/or providing the patient with a treatment plan.

In some embodiments, the system 100 can include software capable of analyzing the patient's genetic testing data along with other patient-specific information (such as patient demographics, medical history, physical location, occupational constraints, etc.), determining genetic mutations, polymorphisms, deletions, insertions, and/or other genetic or epigenetic features of interest from the patient genetic testing data, and/or providing a suggested diagnosis and/or treatment plan, or a mechanism for triggering a suggested diagnosis and/or treatment plan, based on the determination, to the genetic counselor or other care provider of the patient. The suggested diagnosis and/or treatment plan can comprise identification of an existing disease, likelihood of future disease, pre-conception or post-conception advice regarding likelihood of medical characteristics of offspring, identification of family relationships, and/or other useful information. In other embodiments, the software is capable of analyzing genetic data from prospective parents to identify and advise about any recessive disease-related genes that each parent may carry without having any clinical evidence of the disease, but that could result in significant risk of illness in their offspring.

In some embodiments, the centralized computing system can be updated with new or updated software, genetic analysis tools, and/or genetic analysis methods. The patient genetic (inclusive of epigenetic data) testing data can then be re-analyzed using the new or updated software, tools, or methods. The genetic testing data can also be re-analyzed if/when available databases or knowledgebases are updated, such as when a new study or report is published. Based on the re-analysis, the suggested diagnoses and/or treatment plans can be revised. This can be particularly important as new technology is developed in the future for better interpreting genetic testing data, especially for newly recognized genetic features, such as sequence information that are found to correlate with significant health concerns or conditions.

At block 207, the central server 101 can facilitate remote communications between the patient and the genetic counselor, referring physician, or specialist regarding the patient genetic testing results, any diagnoses, and any proposed treatment plans. These communications can similarly comprise remote communications using the system 100 wherein the communications are made using the client devices 102, 103, 104, 108 via the central server 101. All of the descriptions herein relating to other remote communications involving the patient can also apply to these communications involving the patient. For example, such communications can be, encrypted, recorded, transcribed, stored, edited, annotated, and/or later accessed and reviewed.

In some embodiments, the system 100 can store and provide to the patients education materials that the patients can view to learn more about their particular genetic conditions, associated risks, and/or treatment plans. Educational materials can include any format, including text, audio, and/or video materials. The educational material provided can be custom tailored to the patient's language preference, educational background, or other individual preferences. The system 100 can allow the patients to score or grade the educational materials, be tested about materials to assess comprehension of the genetic condition, and/or provide comments or other feedback on the educational materials. The system 100 can then organize and prioritize the educational materials such that the most relevant and useful materials are suggested for each particular patient. The system 100 can also delete and/or edit educational materials that receive poor scores or negative feedback from patients, or which are found to be poorly comprehended.

In some embodiments, the system 100 can facilitate communications between different patients regarding their medical situations. Such patient-patient communications can be remote, using two or more patient clients 103 that connect via the central server. In other embodiments, such patient-patient communications can be non-live, such as via chat rooms or message boards for example. In some embodiments, the patients can be de-identified and/or anonymous during the patient-patient communications. Patient-patient communications can be used to share resources, educational materials, recommendations, advice, emotional support, etc.

At block 208, the patient genetic testing data, reports, diagnoses, treatment plans, and/or other information related to the patient's case can be stored in the patient's electronic medical record in the data warehouse 102. As with other stored information, this stored information can be stored in a secured and/or encrypted format to prevent unauthorized access. The data can be stored in one or more data formats, as discussed above. The data formats can be selected to be human readable, machine readable, digitally parsable, searchable, editable, sharable, scalable, and/or updatable.

In some embodiments, stored information relating to a plurality of different patients can be combined and correlated such that it can later be accessed, mined, and/or analyzed to help better evaluate and treat future patients. Anonymous patient information can be shared with other databases to facilitate the assembly of meta-databases from which greater statistical significance and clinical predictive power can be obtained. For example, certain treatment plans can be evaluated to determine how effective they have been for past patients when treating certain conditions. Also, these data can be used to provide more accurate risk estimations for particular genetic conditions.

In some embodiments, the central server 101 can provide billing related data and automated billing procedures following the patient care related activities to an insurance company or other third-party payer, such as via the insurance/payer client 107. Billing can be automated, payments received can be tracked, automated reminders can be sent, etc.

Any of the data and/or decisions created or obtained during the disclosed methods or by the disclosed systems can be recorded and logged, and can therefore be audited, used for educations studies, used for corrective understanding to help learn from mistakes, used for legal standings such as to show that a diagnosis was correct or appropriate, or used for any other useful purpose.

The genetic testing can include many different forms of testing, including but not limited to the analysis of chromosomes (DNA), proteins, protein expression, DNA methylation, histone modification, and certain metabolites to detect heritable disease-related genotypes, mutations, phenotypes, karyotypes, or other epigenetic modification of genes or chromosomes for clinical purposes. Genetic testing can provide information about a person's genes and chromosomes at various stages in life for various purposes. Exemplary types of genetic testing can include but are not limited to:
1. Newborn screening: Newborn screening can be used just after birth to identify genetic disorders that can be treated early in life. For example, infants can be tested for phenylketonuria and congenital hypothyroidism.
2. Diagnostic testing: Diagnostic testing can be used to diagnose or rule out a specific genetic or chromosomal condition. Genetic testing can be used to confirm a diagnosis when a particular condition is suspected based on physical mutations and symptoms. The results of a diagnostic test can influence a patient's choices about health care and the management of the disease.
3. Carrier testing: Carrier testing can be used to identify people who carry one copy of a gene mutation that, when present in two copies, causes a genetic disorder. This type of testing can be offered to individuals who have a family history of a genetic disorder and to people in ethnic groups with an increased risk of specific genetic conditions, for example. If both parents are tested, the test can provide information about a couple's risk of having a child with a genetic condition such as cystic fibrosis.
4. Pre-implantation genetic diagnosis: This can include genetic testing procedures that are performed on human embryos prior to the implantation as part of an in vitro fertilization procedure.
5. Prenatal diagnosis: Prenatal testing can be used to detect changes in a fetus's genes or chromosomes before birth. This type of testing can be offered to couples with an increased risk of having a baby with a genetic or chromosomal disorder.
6. Predictive and presymptomatic testing: Predictive and presymptomatic types of testing can be used to detect gene mutations associated with disorders that appear after birth, often later in life. These tests can be helpful to people who have a family member with a genetic disorder, but who have no features of the disorder themselves at the time of testing. Predictive testing can identify mutations that increase a person's chances of developing disorders with a genetic basis, such as certain types of cancer. For example, an individual with a mutation in BRCA1 can have a high cumulative risk of breast cancer. Presymptomatic testing can determine whether a person will develop a genetic disorder, such as hemochromatosis, before any signs or symptoms appear. The results of predictive and presymptomatic testing can provide information about a person's risk of developing a specific disorder and help with making decisions about medical care.
7. Pharmacogenomics: This type of genetic testing can determine the influence of genetic variation on drug response.
8. Forensic testing: Forensic testing can utilize DNA sequences to identify an individual for legal purposes. This type of testing can identify crime or catastrophe victims, rule out or implicate a crime suspect, or establish biological relationships between people (for example, paternity).
9. Parental testing: This type of genetic testing can use special DNA markers to identify the same or similar inheritance patterns between related individuals. Because humans inherit half of their DNA from the father and half from the mother, individuals can be tested to find the match of DNA sequences at some highly differential markers to draw a conclusion of relatedness.
10. Genealogical DNA testing: This type of genetic testing can be used to determine ancestry or ethnic heritage for genetic genealogy, for example.
11. Epigenetic testing: As some changes to either chromatin (DNA plus chromosomal proteins) or direct DNA modifications that do not change the coding sequence per se, can influence the ‘read-out’ of genes and therefore contribute risk to individuals, prenatally and/or postnatally. Some epigenetic changes are ‘imprinted’ and conveyed to offspring from either parent(s) through epigenetic modifications of either sperm or egg, these testing measures may detect alterations that place patients, neonates or fetuses at risk.

Any of the disclosed methods may also include recommending and/or performing a patient specific treatment or intensified serial screenings based on the patient's genetic testing data and their other medical and personal information. Such treatments can include surgical treatments, pharmaceutical treatments, nutritional treatments, lifestyle modifications, and/or other types of treatments.

Surgical treatments can include, for example, removal of tissue or organs to reduce/eliminate the risk of future related diseases. For example, breast tissue, ovaries, and/or other reproductive organs may be removed where the genetic testing data and/or personal information indicates a significant risk for cancer or other diseases related to those organs.

Pharmaceutical treatments can include, for example, administering a drug to the patient that is intended to inhibit a condition that the patient is predisposed to develop. For example, statins may be administered to a patient where the patient's genetic testing data and/or other personal information indicate that the patient is likely to develop hyperlipidemia.

Nutritional treatments can include prescribing that patient eat or drink certain foods or consume dietary supplements and/or not eat or drink certain foods or substances, or that they substantially limit dietary intake of certain categories of nutrients. For example, genetic testing data may indicate that a patient has or may develop an abnormality in an enzyme (such as the MTHFR enzyme) for metabolizing vitamin B 12 or folate. In such cases, nutritional treatments may include prescribing methylfolate or methylcobalamin.

Treatments may also include lifestyle modifications based on the genetic testing data. For example, where genetic testing data indicates the patient has a significant risk of birth defects or other problems in becoming pregnant, giving birth, etc., a patient may be advised to carefully consider their reproductive options. In another example, where genetic testing data indicates that a patient is APOE4 positive, advice can be given to avoid head injuries, for example to avoid participating in football or other sports that risk head injuries.

In another example, an aggregate genetic risk score, involving summating the various SNP in an individual, may indicate great risk for the later emergence of neurologic disease, e.g., Parkinson's or Alzheimer's disease, the medical management recommendations may more frequent serial screening (neuroimaging, neurocognitive assessments, plasma proteomic/metabolomics) to gauge the temporal proximity of the disease(s) manifesting themselves. In addition, such proximity measures would allow healthcare professionals to institute an optimal regimen for these individuals a great at-risk which may include marked dietary changes, pharmaceuticals to lower rate of progression to manifest disease and also identify those individuals at greatest risk and offer opportunities for participation in clinical trials using potential disease modifying investigational pharmaceuticals.

In exemplary medical genetics diagnostic evaluation, each patient undergoes a diagnostic evaluation tailored to their own particular presenting signs and symptoms. A geneticist may establish a differential diagnosis and recommend appropriate testing. For example, clinicians may use SimulConsult paired with the National Library of Medicine Gene Review articles to narrow the list of hypotheses (known as the differential diagnosis) and identify the tests that are relevant for a particular patient. These tests might evaluate for chromosomal disorders, inborn errors of metabolism, or single gene disorders.

In some methods, chromosome studies can used to determine a cause for developmental delay/mental retardation, birth defects, dysmorphic features, and/or autism. Chromosome analysis is also performed in the prenatal setting to determine whether a fetus is affected with aneuploidy or other chromosome rearrangements. Additionally, chromosome abnormalities can often be detected in cancer samples. A large number of different methods have been developed for chromosome analysis, including:

1. Chromosome analysis using a karyotype, which involves special stains that generate light and dark bands, allowing identification of each chromosome under a microscope.

2. Fluorescence in situ hybridization (FISH), which involves fluorescent labeling of probes that bind to specific DNA sequences, used for identifying aneuploidy, genomic deletions or duplications, characterizing chromosomal translocations and determining the origin of ring chromosomes.

3. Chromosome painting, which uses fluorescent probes specific for each chromosome to differentially label each chromosome. This technique is more often used in cancer cytogenetics, where complex chromosome rearrangements can occur.

4. Array comparative genomic hybridization, which is a molecular technique that involves hybridization of an individual DNA sample to a glass slide or microarray chip containing molecular probes (e.g., ranging from large ˜200 kb bacterial artificial chromosomes to small oligonucleotides) that represent unique regions of the genome. This method is particularly sensitive for detection of genomic gains or losses across the genome but may not detect balanced translocations or distinguish the location of duplicated genetic material (for example, a tandem duplication versus an insertional duplication).

5. Genome-wide and gene or locus-specific epigenetic analysis. These methods would measure alterations in DNA modifications, e.g., cytosine methylation or others, across all DNA or a specific region of DNA or gene. In addition, chromosomal protein analyses focused on certain chromatin regions may disclose alterations to histone or non-histone proteins that indicate possible defects in gene ‘read-outs’.

Some methods can include basic metabolic studies. In some methods, biochemical studies are performed to screen for imbalances of metabolites in the bodily fluid, usually the blood (plasma/serum) or urine, but also in cerebrospinal fluid (CSF). Specific tests of enzyme function (either in leukocytes, skin fibroblasts, liver, or muscle) may also be employed under certain circumstances. In some cases, a newborn screen incorporates biochemical tests to screen for treatable conditions such as galactosemia and phenylketonuria (PKU). Patients suspected to have a metabolic condition might undergo the various tests, including:

1. Quantitative amino acid analysis, which is typically performed using the ninhydrin reaction, followed by liquid chromatography to measure the amount of amino acid in the sample (either urine, plasma/serum, or CSF). Measurement of amino acids in plasma or serum can be used in the evaluation of disorders of amino acid metabolism such as urea cycle disorders, maple syrup urine disease, and PKU. Measurement of amino acids in urine can be useful in the diagnosis of cystinuria or renal Fanconi syndrome as can be seen in cystinosis.

2. Urine organic acid analysis, which can be either performed using quantitative or qualitative methods, but in either case the test is used to detect the excretion of abnormal organic acids. These compounds are normally produced during bodily metabolism of amino acids and odd-chain fatty acids, but accumulate in patients with certain metabolic conditions.

3. The acylcarnitine combination profile detects compounds such as organic acids and fatty acids conjugated to carnitine. The test is used for detection of disorders involving fatty acid metabolism, including MCAD.

4. Pyruvate and lactate are byproducts of normal metabolism, particularly during anaerobic metabolism. These compounds normally accumulate during exercise or ischemia, but are also elevated in patients with disorders of pyruvate metabolism or mitochondrial disorders.

5. Ammonia is an end product of amino acid metabolism and is converted in the liver to urea through a series of enzymatic reactions termed the urea cycle. Elevated ammonia can therefore be detected in patients with urea cycle disorders, as well as other conditions involving liver failure.

6. Enzyme testing, which can be performed for a wide range of metabolic disorders to confirm a diagnosis suspected based on screening tests.

Some methods can include molecular studies. For example, some methods can include one or more of the following examples:

1. DNA sequencing, which is used to directly analyze the genomic DNA sequence of a particular gene. In general, only the parts of the gene that code for the expressed protein (exons) and small amounts of the flanking untranslated regions and introns are analyzed.

2. DNA methylation analysis, which is used to diagnose certain genetic disorders that are caused by disruptions of epigenetic mechanisms such as genomic imprinting and uniparental disomy.

3. Southern blotting, which is an early technique basic on detection of fragments of DNA separated by size through gel electrophoresis and detected using radiolabeled probes. This test was routinely used to detect deletions or duplications in conditions such as Duchenne muscular dystrophy but is being replaced by high-resolution array comparative genomic hybridization techniques. Southern blotting is still useful in the diagnosis of disorders caused by trinucleotide repeats.

4. Short tandem repeats, which are unique markers that can be used to determine haplotypes and are used in identity testing for maternal cell contamination.

A patient can be treated with a variety of different treatment option based on the results of the genetic testing for that patient, their other personal information, and/or other data. Since genetic syndromes are often the result of alterations of the chromosomes or genes, there may be no treatment that can correct the genetic alterations in every cell of the body. However, for many genetic syndromes there are treatment options available to manage the symptoms. In some cases, particularly inborn errors of metabolism, the mechanism of disease is well understood and offers the potential for dietary and medical management to prevent or reduce the long-term complications. In other cases, infusion therapy can be used to replace the missing enzyme. In some cases, gene therapy can be used to treat specific genetic disorders.

Metabolic disorders can arise from enzyme deficiencies that disrupt normal metabolic pathways. For instance, in the hypothetical example, compound A is metabolized to B by enzyme X, compound B is metabolized to C by enzyme Y, and compound C is metabolized to D by enzyme Z. If enzyme Z is missing, compound D will be missing, while compounds A, B, and C will build up. The pathogenesis of this particular condition could result from lack of compound D, if it is critical for some cellular function, or from toxicity due to excess A, B, and/or C. Treatment of the metabolic disorder may be achieved through dietary supplementation of compound D and dietary restriction of compounds A, B, and/or C or by treatment with a medication that promoted reduction of excess A, B, or C as these may be toxic to patients. Another approach that can be taken is enzyme and/or gene replacement therapy, in which a patient is given an infusion of the missing enzyme.

Dietary or nutritional treatments can also be utilized. For example, dietary restriction and supplementation can be useful in treating several metabolic disorders, including galactosemia, phenylketonuria (PKU), maple syrup urine disease, organic acidurias, and urea cycle disorders. Such restrictive diets can be difficult for the patient and family to maintain, and may require close consultation with a nutritionist who has special experience in metabolic disorders. The composition of the diet may change depending on the caloric needs of a growing child, for example, and special attention may be needed during a pregnancy if a woman is affected with such a disorder.

Medication or pharmaceutical treatments can include, for example, enhancement of residual enzyme activity (in cases where the enzyme is made but is not functioning properly), inhibition of other enzymes in the biochemical pathway to prevent buildup of a toxic compound, or diversion of a toxic compound to another form that can be excreted. Examples include the use of high doses of pyridoxine (vitamin B6) in some patients with homocystinuria to boost the activity of the residual cystathione synthase enzyme, administration of biotin to restore activity of several enzymes affected by deficiency of biotinidase, treatment with NTBC in Tyrosinemia to inhibit the production of succinylacetone which causes liver toxicity, and the use of sodium benzoate to decrease ammonia build-up in urea cycle disorders.

Enzyme and/or gene replacement therapy is another treatment option. For example, certain lysosomal storage diseases can be treated with infusions of a recombinant enzyme (e.g., produced in a laboratory), which can reduce the accumulation of the compounds in various tissues. Examples include Gaucher disease, Fabry disease, Mucopolysaccharidoses and Glycogen storage disease type II. Such treatments may be limited by the ability of the enzyme to reach the affected areas (the blood brain barrier prevents enzyme from reaching the brain, for example), and can sometimes be associated with allergic reactions. Recent developments with gene therapy make systemic, visceral or central nervous system organ gene replacement therapy another therapeutic option, depending on the nature of patient defect and which body compartments are most affected by the loss of enzyme function.

Other treatment options can include angiotensin receptor blockers in Marfan syndrome and Loeys-Dietz, bone marrow transplantation, and gene therapy.

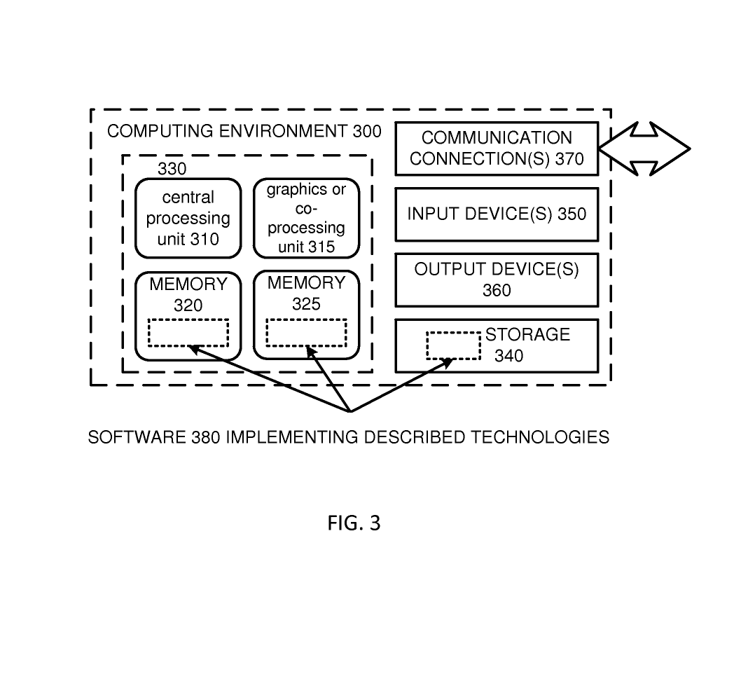

FIG. 3 depicts a generalized example of a suitable computing environment 300 in which the described technology may be implemented. The computing environment 300 is not intended to suggest any limitation as to scope of use or functionality, as the technology may be implemented in diverse general-purpose or special-purpose computing systems. For example, the computing environment 300 can include any one or more of a variety of computing devices (e.g., desktop computer, laptop computer, server computer, tablet computer, mobile device, etc.). Further, the computing environment 300 can comprise any number of connected devices that work together and/or independently to perform the disclosed technology. In some embodiments, a centralized computing system includes one or more server computers and one or more databases that function together. The overall network can include any number of client computing devices that communicate remotely with the server computers or other aspects of the computing environment 300. Such client devices can include a patient's or physician's home computer, work computer, or mobile device, for example.

Electronic applications, such as those disclosed herein, can run on a first electronic device, and communicate with another electronic device in a remote or distributed manner, in some embodiments. A web application can also be used instead of or in conjunction with an application on user's device. For example the data can be received from a user at the user's computing device, through an application or on a website, and some of the data or other data based on the user input data can be communicated over the Internet or wirelessly with a remote server or other computer to provide part of the functionality of the application.

With reference to FIG. 3, the computing environment 300 includes one or more processing units 310, 315 and memory 320, 325. In FIG. 3, this basic configuration 330 is included within a dashed line. The processing units 310, 315 execute computer-executable instructions. A processing unit can be a general-purpose central processing unit (CPU), processor in an application-specific integrated circuit (ASIC) or any other type of processor. In a multi-processing system, multiple processing units execute computer-executable instructions to increase processing power. For example, FIG. 3 shows a central processing unit 310 as well as a graphics processing unit or co-processing unit 315. The tangible memory 320, 325 may be volatile memory (e.g., registers, cache, RAM), non-volatile memory (e.g., ROM, EEPROM, flash memory, etc.), or some combination of the two, accessible by the processing unit(s). The memory 320, 325 stores software 380 implementing one or more innovations described herein, in the form of computer-executable instructions suitable for execution by the processing unit(s).

A computing system may have additional features. For example, the computing environment 300 includes storage 340, one or more input devices 350, one or more output devices 360, and one or more communication connections 370. An interconnection mechanism (not shown) such as a bus, controller, or network interconnects the components of the computing environment 300. Typically, operating system software (not shown) provides an operating environment for other software executing in the computing environment 300, and coordinates activities of the components of the computing environment 300.

The tangible storage 340 may be removable or non-removable, and includes magnetic disks, magnetic tapes or cassettes, CD-ROMs, DVDs, or any other medium which can be used to store information in a non-transitory way and which can be accessed within the computing environment 300. The storage 340 stores instructions for the software 380 implementing one or more innovations described herein.

The input device(s) 350 may be a touch input device such as a keyboard, mouse, pen, or trackball, a voice input device, a scanning device, or another device that provides input to the computing environment 300. For video recording or encoding, the input device(s) 350 may be a camera, video card, TV tuner card, or similar device that accepts video input in analog or digital form, or a CD-ROM or CD-RW that reads video samples into the computing environment 300. The output device(s) 360 may be a display, printer, speaker, CD-writer, or another device that provides output from the computing environment 300.

The communication connection(s) 370 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

Any of the disclosed methods can be implemented as computer-executable instructions stored on one or more computer-readable storage media (e.g., one or more optical media discs, volatile memory components (such as DRAM or SRAM), or nonvolatile memory components (such as flash memory or hard drives)) and executed on a computer (e.g., any commercially available computer, including smart phones or other mobile devices that include computing hardware). The term computer-readable storage media does not include communication connections, such as signals and carrier waves. Any of the computer-executable instructions for implementing the disclosed techniques as well as any data created and used during implementation of the disclosed embodiments can be stored on one or more computer-readable storage media. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, the disclosed technology can be implemented by software written in C++, Java, Perl, Ruby, JavaScript, Adobe Flash, or any other suitable programming language. Likewise, the disclosed technology is not limited to any particular computer or type of hardware. Certain details of suitable computers and hardware are well known and need not be set forth in detail in this disclosure.

It should also be well understood that any functionality described herein can be performed, at least in part, by one or more hardware logic components, instead of software. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means.

The disclosed methods, apparatus, and systems should not be construed as limiting in any way. Instead, the present disclosure is directed toward all novel and nonobvious features and aspects of the various disclosed embodiments, alone and in various combinations and subcombinations with one another. The disclosed methods, apparatus, and systems are not limited to any specific aspect or feature or combination thereof, nor do the disclosed embodiments require that any one or more specific advantages be present or problems be solved.

Although the operations of some of the disclosed methods are described in a particular, sequential order for convenient presentation, it should be understood that this manner of description encompasses rearrangement, unless a particular ordering is required by specific language set forth herein. For example, operations described sequentially may in some cases be rearranged or performed concurrently. Moreover, for the sake of simplicity, the attached figures may not show the various ways in which the disclosed methods can be used in conjunction with other methods.

As used in this application and in the claims, the singular forms “a,” “an,” and “the” include the plural forms unless the context clearly dictates otherwise. Additionally, the term “includes” means “comprises.” Further, the term “coupled” generally means electrically, wirelessly, and/or physically coupled or linked and does not exclude the presence of intermediate elements between the coupled items absent specific contrary language.

In view of the many possible embodiments to which the principles disclosed herein may be applied, it should be recognized that the illustrated embodiments are only preferred examples and should not be taken as limiting the scope of the technology. Rather, the scope of the disclosure is at least as broad as the following claims.

1. A method for telegenetics consultation, comprising:
providing a centralized computing system comprising at least one medical information management computer including a communication medium to communicate with remote sites and memory to store data thereon to thereby define a remote medical services server;
providing a database associated with the remote medical services server and including a plurality of patient electronic medical records providing a single consolidated medical genetic information record for at least one of a corresponding plurality of patients, the electronic medical records accessible by a patient clinic medical service provider, a medical services scheduler, and a remote genetic counselor, wherein the medical services server:
receives a referral of a patient for genetic counseling from a referring physician or clinician associated with the patient clinic medical service provider;
receives the medical genetic information record that contains patient medical data relevant for patient genetic counseling and provides the patient medical data to a genetic counselor;
facilitates remote communication between the patient and the genetic counselor regarding possible genetic testing and counseling;
receives a genetic testing data requisition from the genetic counselor or the referring physician indicating a request to obtain genetic testing data for the patient;
obtains genetic testing data for the patient;
provides the patient electronic medical record containing the patient genetic testing data to the genetic counselor;
facilitates remote communication between the patient and one or more of the genetic counselor, the referring physician, and one or more specialists regarding the patient genetic testing data and a proposed treatment plan; and
stores the patient genetic testing data and the proposed treatment plan in the electronic medical record in the database.

2. The method of claim 1, wherein the medical services server captures and stores in the electronic medical record communication data from the remote communications in an encrypted, secure format that allows subsequent searching and analysis of the stored communication data.

3. The method of claim 2, wherein at least some of the stored communication data in the electronic medical record can be reviewed, corrected, and clarified by one or more of the patient, the referring physician, the genetic counselor, and the specialists.

4. The method of claim 1, wherein the remote communications include voice communications and the medical services server translates and transcribes the voice communications and stores them in the electronic medical record.

5. The method of claim 1, wherein at least some of the remote communications are facilitated via VoIP.

6. The method of claim 1, wherein the patient genetic testing data includes variant data via variant call format files.

7. The method of claim 1, wherein the patient genetic testing data includes curated genetic testing reports.

8. The method of claim 1, wherein the medical services server:
analyzes the patient genetic testing data and the patient medical data;
provides a mechanism for triggering a suggested treatment plan for the patient to the genetic counselor or the referring physician based on the patient genetic testing data and the patient medical data; and
stores the suggested treatment plan in the electronic medical record.

9. The method of claim 1, wherein the medical services server provides an electronic visual representation of the patient genetic testing data that indicates relevant mutations in interrogated genes of the patient.

10. The method of claim 1, wherein the database contains current genetic medicine literature, and the medical services server provides access to the genetic medicine literature to one or more of the referring physician, the genetic counselor, and the specialists.

11. The method of claim 1, wherein the medical services server receives scheduling information from the patient and from one or both of the referring physician and the genetic counselor, and automatically schedules the remote communications based on mutual scheduling availabilities.

12. The method of claim 1, wherein the medical services server provides educational content to the patient regarding genetics and the patient genetic testing data, wherein the educational content is based on patient-specific characteristics and input from the genetic counselor.

13. The method of claim 1, wherein the medical services server provides for anonymous, remote communications between the patient and other patients with similar genetic conditions.

14. The method of claim 1, wherein the medical services server facilitates scheduling of medical facilities for genetic medicine services based on locations of the facilities and services available at the facilities.

15. The method of claim 1, wherein the medical services server:
provides a synthetic avatar that is displayed to the patient at a patient location remote from the server and database, wherein the synthetic avatar asks the patient questions regarding conditions relevant to genetic counseling;
collects data provided by the patient in response to the questions; and
stores the collected data in the database in a secure, encrypted format for subsequent review.

16. The method of claim 1, wherein the centralized computing system:
analyzes the patient genetic testing data;
determines genetic mutations, polymorphisms, deletions, insertions, or other genetic features of interest from the patient genetic testing data;
reports said determination; and
provides a suggested diagnosis and treatment plan for the patient based on said determination.

17. The method of claim 16, wherein the suggested diagnosis comprises identification of an existing disease, likelihood of future disease, pre-conception advice regarding likelihood of medical characteristics of offspring, or identification of family relationships.

18. The method of claim 16, further comprising:
updating the centralized computing system with new or updated genetic analysis tools or genetic analysis methods;
re-analyzing the patient genetic testing data using the new or updated genetic analysis tools or genetic analysis methods; and
revising the suggesting diagnosis and suggested treatment plan based on the re-analyzed patient genetic testing data.

19. The method of claim 1, wherein the medical services server facilitates remote communication between the genetic counselor and the referring physician regarding patient evaluation and recommendations for genetic testing, prior to genetic testing.

20. The method of claim 1, wherein the medical services server facilitates live, remote communication between two or more of the genetic counselor, the referring physician, and specialists regarding creating a proposed treatment plan for the patient based on the patient medical data and the patient genetic testing data.

21. The method of claim 1, wherein the medical services server provides medical billing data for an insurance provider or other payer based on telegenetics services provided for the patient.

22. The method of claim 1, wherein the medical services server receives genetic testing data for the patient by:
providing the genetic testing data requisition to a molecular diagnostic laboratory; and
receiving patient genetic testing data from the molecular diagnostic laboratory, the patient genetic testing data resulting from genetic testing for the patient performed by the molecular diagnostic laboratory in response to the requisition.

23. The method of claim 1, wherein the remote communication is live, real-time communication.

24. The method of claim 1, wherein the genetic counselor is a certified genetic specialist.

25. The method of claim 1, further comprising treating the patient based at least in part on the patient genetic testing data, wherein the treating comprises one or more of a surgical treatment, a pharmaceutical treatment, a nutritional treatment, and a lifestyle modification.

26. One or more computer-readable storage devices comprising computer-readable instructions for causing an electronic computing device or system to perform the method of claim 1.

27. A system for telegenetics consultation, the system comprising a centralized computing system having at least one central server or remote medical services server, and at least one data warehouse or database, wherein the system is configured to perform the method of claim 1.

28. The system of claim 27, further comprising a patient client computing device located remote from the centralized computing system and configured to facilitate remote communications between the patient and one or more of the referring physician, the genetic counselor, and the one or more specialists.

29. The system of claim 27, further comprising a referring physician client computing device located remote from the centralized computing system and configured to facilitate remote communications between the referring physician and one or more of the client, the genetic counselor, and the one or more specialists.

30. The system of claim 27, further comprising a genetic counselor client computing device located remote from the centralized computing system and configured to facilitate remote communications between the genetic counselor and one or more of the client, the referring physician, and the one or more specialists.

31. The system of claim 28, wherein one or more of the patient client computing device, the referring physician client computing device, and the genetic counselor client computing device, comprises a camera and voice recorder configured to facilitate live video and audio communication between two or more of the patient, the referring physician, the genetic counselor, and the one or more specialists.
